(19) United States
(12) Patent Application Publication
(10) Pub. No.: US 2024/0408742 A1
(43) Pub. Date: Dec. 12, 2024

(54) Exoskeletons Comprising Flexible Mechanisms

(71) Applicants: suitX, Inc., Emeryville, CA (US); Ottobock SE & Co. KGaA, Duderstadt (DE)

(72) Inventors: James Hatch, Oakland, CA (US); John Kuwata, El Cerrito, CA (US); Minerva Pillai, Redwood City, CA (US); Maximiliam Merker, Goettingen (DE); Michael Bremer, Goettingen (DE); Carsten Vogel, Duderstadt (DE); Fabienne Röschel, Goettingen (DE); Oliver Mizera, Goettingen (DE); Matthias Vollbrecht, Herzberg am Harz (DE); Pascal Schwedhelm, Duderstadt (DE); Laura Hoffsümmer, Duderstadt (DE)

(21) Appl. No.: 18/811,997

(22) Filed: Aug. 22, 2024

Related U.S. Application Data
(63) Continuation of application No. PCT/US23/17054, filed on Mar. 31, 2023.
(60) Provisional application No. 63/362,382, filed on Apr. 1, 2022.

(51) Int. Cl. B25J 9/00

(52) U.S. Cl. CPC B25J 9/0006

(57) ABSTRACT

A flexible mechanism for an exoskeleton allows for a simple and low-cost method of allowing a high range of motion between an frame and a human interface while effectively transferring support forces from the frame to the human interface. Additionally, the flexible mechanism adds kinematic redundancy that compensates for incongruities between the exoskeleton and human during forward flexion to prevent the human interface from uncomfortably sliding on the person via purely tension-loaded force transmitting elements.

### CROSS-REFERENCE TO RELATED APPLICATIONS

This application is a continuation of PCT/US2023/017054 filed on 2023 Mar. 31 and published as WO2023/192572 on 2023 Oct. 5, which claims the benefit under 35 U.S.C. § 119 (e) of U.S. Provisional Patent Application 63/362,382, filed on 2022 Apr. 1, which is incorporated herein by reference in its entirety for all purposes.

### FIELD OF TECHNOLOGY

The present disclosure pertains to the art of exoskeletons to support the human body, particularly exoskeletons configured to reduce the bending moment on a person's back during a forward bend.

### BACKGROUND

Exoskeletons are used to assist people in bending, standing upright, or any combination of these movements. Conventional exoskeletons are designed such that a moment is created during a bend to counteract the moments from a person's trunk gravity weight. These exoskeletons may utilize passive spring resistance or active motor resistance to create a torque between the wearer's trunk and legs. Some exoskeletons may also utilize a rigid frame with defined rotation axes, or a flexible interface to apply the supportive torque to the person such that it acts about the person's hip or lower back to reduce the probability of injury to the spine.

To comfortably apply the supportive torque, an exoskeleton must move in tandem with both the thighs and the trunk of the user and minimize relative motion between the exoskeleton and the person that would otherwise cause rubbing, chafing, or general discomfort. In one conventional exoskeleton, the alignment between the person's hip joint and the exoskeleton hip joint is used to help achieve this, as well as a human interface slidable along an axis substantially parallel with the spine of the user to help facilitate bending maneuvers. While these solutions are helpful to minimize relative motion between the exoskeleton and the person during bending motions, they remain inadequate to compensate for the complex structure and movement of the spine as well as inevitable misalignments of the person's hip joint and exoskeleton hip joint due to errors in the fitting.

In addition to creating the supporting torque about the hips and lower back, a secondary aim of the exoskeleton is to allow free movement of the person's trunk during non-bending motions such as twisting, side bending, or walking. Other exoskeletons used mechanical joints with axes orthogonal to the exoskeleton hip joint that allow twisting and side bending motions of the person's trunk while still allowing for the transfer of a supportive torque during forward bending. Again, because of the complex structure and movement of the spine, these mechanical joints can not perfectly align with the biological axes of rotation, causing rubbing, chafing, or general discomfort. When walking, both the person's hips and spine move in a manner to maximize the efficiency of gait. To best allow for walking, an exoskeleton must allow for the relative motion between the hips and trunk in the frontal and transverse planes, again with the closest alignment of the biological and mechanical joints as possible. If the exoskeleton does not provide the secondary degrees or freedom necessary for a person s gait, increased energy expenditure during walking or rubbing, chafing, and general discomfort at the exoskeleton contact points may occur.

### SUMMARY

The present disclosure is directed to an exoskeleton, which is configured to be worn by a wearer to reduce the muscle forces in the wearer's back during forward lumbar flexion. In some embodiments, the exoskeleton comprises: a frame, which is configured to be coupled to the wearer's trunk; two links, which are configured to move in unison with the wearer's thighs in a manner resulting in flexion and extension of respective links relative to the frame; and two torque generators located on both left and right halves of the wearer substantially close to the wearer's hip. The torque generators couple the frame to the respective links and are configured to generate torque between the links and the frame. The exoskeleton may include active or passive means for actuating the torque generators. In operation when the wearer bends forward in the sagittal plane such that a predetermined portion of the frame passes beyond a predetermined angle from the vertical gravity line, at least one of the first or second torque generators imposes a resisting torque between the frame and at least one of the links. This causes the frame to impose a force against the wearer's trunk and at least one of the links to impose a force onto the wearer's thigh. The torque generators may be activated or deactivated based on the inclination angle of the person's trunk or other inputs not described.

The present disclosure is directed to a flexible mechanism configured to couple a frame to a human interface. In some embodiments, the flexible mechanism is configured to couple a human interface to the frame of an exoskeleton. The flexible mechanism allows for a simple and low-cost method of allowing a high range of motion between the frame and the human interface while effectively transferring supportive forces and torques. The flexible mechanism adds kinematic redundancy that both compensates for incongruities between the exoskeleton and the human during forward flexion and/or allows for secondary motions such as side bending, twisting, and walking that minimizes the human interface from uncomfortably sliding relative to the person. Furthermore, due to the nature of the flexible mechanism, no discrete center of rotation is defined for rotation motions, further allowing for the motion of the human interface to more closely follow the motion of the person's trunk while the frame follows the motion of the person's hips.

In some embodiments, an exoskeleton is configured to be worn by a person to reduce the muscle forces in the person's back during forward lumbar flexion, the exoskeleton comprising, a frame configured to produce or transfer a supporting torque, and a human interface configured to couple to the trunk of the person, the human interface comprising a rigid back plate, and a flexible mechanism coupled the rigid back plate at two locations and loops around, or at least partially encircles, the frame, the flexible mechanism configured to transfer tensile forces between the frame and the human interface, wherein the flexible mechanism restricts a frontal translation motion between the human interface and the frame to transfer the supporting torque from the frame to the human interface through the tensile forces, and the flexible mechanism allows for a sagittal translation motion, transverse translation motion, frontal rotation motion, sagittal rotation motion, and transverse rotation motion between the human interface and the frame. The looped end of the flexible element may be configured to slide relative to the frame.

In some embodiments, when the person bends forward and the frame produces or transfers the supporting torque, the flexible mechanism restricts a frontal translation motion to transfer the supporting torque from the frame to the human interface, and the flexible mechanism allows for a sagittal rotation motion and a transverse translation motion between the frame and the human interface reduce relative motion between the human interface and the person's trunk.

In some embodiments, when the person bends forward and the frame produces or transfers the supporting torque, the flexible mechanism restricts a frontal translation motion to transfer the supporting torque from the frame to the human interface, and the flexible mechanism provides a frontal rotation motion, and a sagittal translation motion to allow the person to side bend.

In some embodiments, when the person bends forward and the frame produces or transfers the supporting torque, the flexible mechanism restricts a frontal translation motion to transfer the supporting torque from the frame to the human interface, and the flexible mechanism provides a transverse rotation motion to allow the person to twist.

In some embodiments, when the person performs a walking motion, the frame follows the motion of the person's hips, and the human interface moves in a sagittal translation motion to follow the motion of the person's trunk.

In some embodiments, the flexible mechanism provides for a range of motion selected from a group consisting of sagittal translation, transverse translation, frontal rotation, and transverse rotation to allow for the person's hips to move relative to the person's trunk during walking.

In some embodiments, the exoskeleton further comprises a cam element coupled to either the human interface or the frame, wherein the cam element is configured to create a space and provide a rolling contact surface between the human interface and the frame to allow for rotation motions.

In some embodiments, the exoskeleton further comprises a centering element coupled between the frame and the human interface, wherein the centering element is configured to apply a force to bring the human interface to a neutral position relative to the frame.

In some embodiments, the human interface further comprises a shoulder strap coupled to the rigid back plate configured to at least partially encircle a person's shoulders or chest. The shoulder strap may be coupled to the rigid back plate at an upper location and a lower location. In some embodiments, the flexible mechanism is located halfway between the upper location and the lower location. In other embodiments, the flexible mechanism is aligned with the lower location.

In some embodiments, an exoskeleton is configured to be worn by a person to reduce the muscle forces in the person's back during forward lumbar flexion, the exoskeleton comprising: a frame configured to produce or transfer a supporting torque, and a human interface configured to couple to the trunk of the person, and a flexible mechanism configured to transfer tensile forces between the frame and the human interface during inclination in the sagittal plane, wherein the connection is freely movable in all other directions.

In other embodiments, an exoskeleton is configured to be worn by a person to reduce muscle forces of the person, the exoskeleton comprises: a frame configured to produce or transfer a supporting torque; an interface configured to couple to the person; and a flexible mechanism configured to transfer tensile forces between the frame and the interface, wherein when the frame produces or transfers the supporting torque, the flexible mechanism transfers the supporting torque to the interface by means of the tensile forces. The flexible mechanism restricts a translation motion between the frame and the interface approximately orthogonal to the segment of the person's body to be supported while allowing free rotation and translation in all other directions. In other embodiments the flexible mechanism restricts a translation motion between the frame and the interface in line with the tensile forces while allowing free rotation and translation motion in all other directions.

This application adds additional utility to exoskeleton devices, particularly exoskeleton devices. These and other embodiments are described further below with reference to the figures.

### BRIEF DESCRIPTION OF THE DRAWINGS

The included drawings are for illustrative purposes and serve only to provide examples of possible structures and operations for the disclosed inventive systems and methods. These drawings in no way limit any changes in form and detail that may be made by one skilled in the art without departing from the spirit and scope of the disclosed implementations.

FIG. 1 depicts a side view of an exoskeleton on a forward bending person, in accordance with some examples.

FIG. 2 depicts a side view of an exoskeleton on a standing person, in accordance with some examples.

FIG. 3 depicts the definition of various planes and motions relative to a person, in accordance with some examples.

FIG. 4 depicts a rear perspective view of an exoskeleton with a flexible mechanism, in accordance with some examples.

FIG. 5 depicts a side view of the exoskeleton with a flexible mechanism on a forward bending person, in accordance with some examples.

FIG. 6 depicts a side view of the flexible mechanism in a sagittal rotation motion, in accordance with some examples.

FIGS. 7a-7c depict a top view of the flexible mechanism in (a) a neutral position (b) a transverse rotation motion and (c) a sagittal translation motion, in accordance with some examples.

FIGS. 8a-8c depict a back view of the flexible mechanism in (a) a neutral position (b) a fontal rotation motion and (c) a transverse translation motion, in accordance with some examples.

FIG. 9 depicts a rear perspective view of an alternate embodiment of an exoskeleton with a flexible mechanism, in accordance with some examples.

FIG. 10 depicts a side view of the exoskeleton with a flexible mechanism on a forward bending person, in accordance with some examples.

FIG. 11 depicts a side view of the flexible mechanism in a sagittal rotation motion, in accordance with some examples.

FIG. 12 depicts a top view of the flexible mechanism in (a) a neutral position (b) a transverse translation motion (c) a sagittal translation motion and (d) a frontal rotation motion, in accordance with some examples.

FIG. 13 depicts a back view of the flexible mechanism in (a) a neutral position (b) transverse rotation motion, in accordance with some examples.

FIG. 14 depicts a rear view of an alternate embodiment of an exoskeleton with a flexible mechanism, in accordance with some examples.

FIG. 15 depicts a rear view of a flexible mechanism in (a) a neutral position (b) sagittal translation motion (c) frontal rotation motion and (d) transverse rotation motion, in accordance with some examples.

FIG. 16 depicts a side view of a flexible mechanism in (a) a neutral position (b) transverse translation motion and (c) sagittal rotation motion, in accordance with some examples.

FIG. 17 depicts a rear view of a flexible mechanism with a self-centering element, in accordance with some examples.

FIG. 18 depicts a side view of an alternate embodiment of a flexible mechanism with a self-centering element, in accordance with some examples.

### DETAILED DESCRIPTION

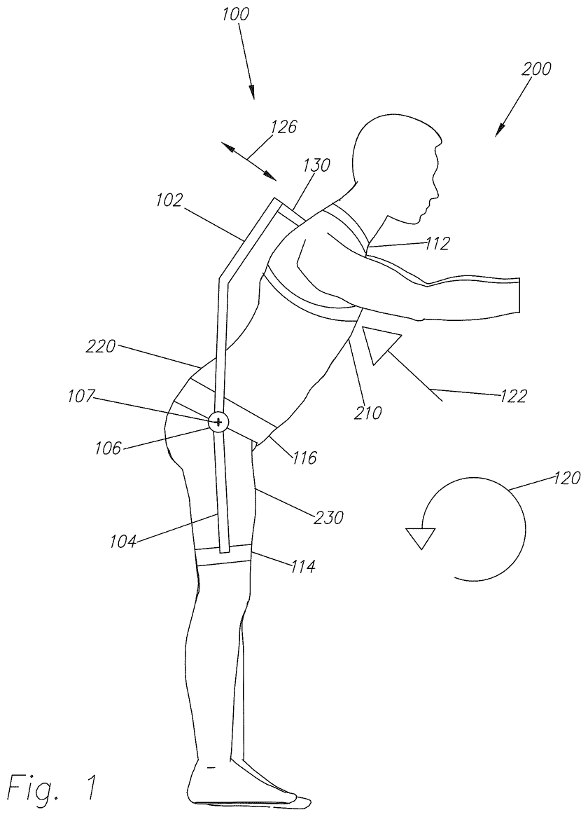

FIG. 1 shows a general embodiment of flexible mechanism 130 for exoskeleton 100 on forward bending person 200. Exoskeleton 100 is configured to be worn by person 200 to reduce the muscle forces during forward bending motions. In general, exoskeleton 100 comprises frame 102 configured to be coupled to person's trunk 210; link 104 configured to move in unison with person's thigh 230 in a manner resulting in flexion and extension of link 104 relative to frame 102; and at least one torque generator 106 configured to generate supporting torque 120 to frame 102. Exoskeleton 100 further comprises a human interface 112 configured to couple frame 102 to person's trunk 210, thigh strap 114 configured to couple link 104 to person's thigh 230, and belt 116 configured to couple the exoskeleton to person's hips 220. Flexible mechanism 130 allows person 200 to move freely while wearing exoskeleton 100, while transferring the support from exoskeleton 100 to the body of person 200. In some embodiments, flexible mechanism 130 is configured to connect a thoracic, spinal, or supporting trunk portion of frame 102 to human interface 112. Flexible mechanism 130 provides a sufficient range of motion for human interface 112 to twist and bend sideways relative to frame 102, yet is able to transfer support torque 120 between frame 102 and human interface 112.

In operation, when person 200 bends forward in sagittal plane 300, torque generator 106 generates supporting torque 120 between link 104 and frame 102. Frame 102 is rigid and transfers supporting torque 120 along the length of frame 102. Flexible mechanism 130 transfers supporting torque 120 from frame 102 to human interface 112 via tensile forces along tensile force direction 126. Human interface 112 then applies supporting force 122 to person's trunk 210, thus reducing back muscle forces required to hold up the weight of person's trunk 210 during bending motions. Flexible mechanism 130 does not elongate along tensile force direction 126 such that it can effectively transfer forces between frame 102 and human interface 112 during forward bending. While remaining rigid along tensile force direction 126, flexible mechanism 130 allows for free motion between frame 102 and human interface 112 in all other directions.

In some embodiments, torque generator 106 is actively powered with energy input into the system and is selected from a group consisting of electric motors, rotary actuators, linear actuators, hydraulics, pneumatics, or artificial muscles. In other embodiments torque generator 106 is passively powered and stores and releases energy and is selected from a group consisting of springs, elastics, rubbers, bungee chords, gas springs, of similar materials. Still, in other embodiments, frame 102 and/or link 104 is made of a resilient material that stores and releases energy thus acting as torque generator 106 if rotation about joint 107 is locked. In this embodiment frame 102 generates or produces supportive torque 120 through elastic deformation. In this embodiment frame 102 may be made of a resilient material such as metal, carbon fiber, or fiberglass. In this embodiment exoskeleton 100 may further comprise a lock between frame 102 and link 104 to prevent their relative rotation about joint 107.

The freedom of motion allowed by flexible mechanism 130 allows person's trunk 210 to twist, side bend, or rotate relative to person's hips 220. This allows person 200 to maneuver their upper body while bending forward to better reach, pick up, or put down objects while receiving supporting force 122 to compensate for the bending motion in sagittal plane 300. Flexible mechanism 130 may comprise one or a combination of flexible elements, semi-rigid elements, or rigid elements configured to allow translation and rotation in all planes except motion corresponding to the forward bending of person 200 in the sagittal plane.

The freedom of motion allowed by flexible mechanism 130 also provides kinematic redundancy to offset misalignments between the axes of rotation of the person's body and the axes of rotation of exoskeleton 100. In some embodiments, a rotational axis between frame 102 and link 104 is designed to approximately cross through the hip joint of person 200. When the rotational axis is properly aligned with person 200, link 104 will move substantially in unison with person's thigh 230 and frame 102 will move substantially in unison with person's trunk 210 throughout the person's forward bending range of motion. If the rotational axis is misaligned with person 200, frame 102 will move relative to person's trunk 210 or link 104 will move relative to person's thigh 230. If flexible mechanism 130 is not present, the movement of frame 102 will cause human interface 112 to slide or rotate on person 200 and create discomfort. When flexible mechanism 130 is present, human interface 112 is able to move relative to frame 102, allowing human interface 112 to remain static on person's trunk 210 while supporting torque 120 is transferred through frame 102, flexible mechanism 130, and human interface 112 to person 200. Flexible mechanism 130 could similarly be used to allow motion between thigh strap 114 and link 104. In another embodiment, the rotational axis is designed to approximately cross through the lower spine of person 200.

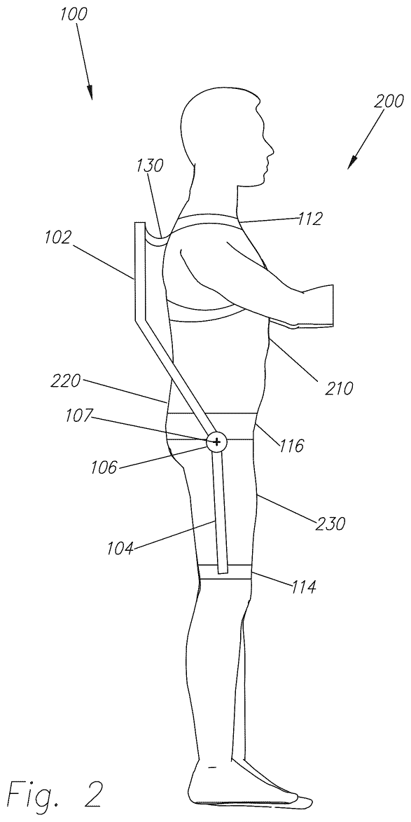

FIG. 2 shows a general embodiment of flexible mechanism 130 for exoskeleton 100 on standing person 200. In this position, exoskeleton 100 is not producing supporting torque 120 and no tensile forces are being transmitted through flexible mechanism 130. Because person 200 is not bending forward, supporting torque 120 is not needed, and slack may be present in flexible mechanism 130. In this position flexible mechanism allows free motion of human interface 112 relative to frame 102 in all directions. The freedom of motion allowed by flexible mechanism 130 allows for person's trunk 210 to twist, bend, or rotate relative to person's hips 220. This allows for person 200 to freely move trunk 210 and upper body relative to hips 220 and lower body to allow many postures and tasks such as standing, walking, and overhead work. In some embodiments, torque generator 106 can be selectively turned off and on. When torque generator 106 is turned off, slack may be present in flexible mechanism 130 for all postures, including forward bending.

When person 200 moves from the position shown in FIG. 2 into a forward bending motion in sagittal plane 300, the slack will be removed from flexible mechanism 130 and tensile forces will be transferred between frame 102 and human interface 112 in tensile force direction 126 according to FIG. 1. The length of flexible mechanism 130 can be adjusted to tune how much the sagittal rotation motion 302 about joint 107 must occur before tensile forces can be transferred between frame 102 and human interface 112. In some embodiments, flexible mechanism 130 is arranged to minimize the amount of slack in the forward bending direction while maintaining freedom of motion in all other directions in order to most efficiently transfer supporting torque 120 from exoskeleton 100 to person 200.

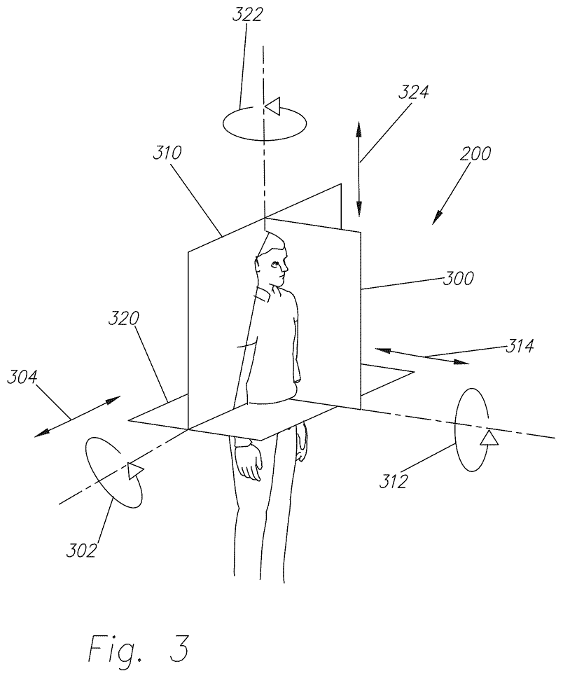

FIG. 3 depicts the definition of various planes and motions relative to person 200. The same planes and motions may similarly describe exoskeleton 100 as if it was worn by person 200. All planes are depicted as crossing through a midpoint of person 200 for clarity but may be shifted to divide the person differently as long as they remain parallel to the depiction shown in FIG. 3. Sagittal plane 300 divides person 200 into right and left portions, and when viewed orthogonally depicts a side view of person 200 or exoskeleton 100. Sagittal rotation motion 302 corresponds to a rotation along an axis orthogonal to sagittal plane 300. Flexion/extension of the person's hips, lower spine, upper spine, or neck may have a component of sagittal rotation motion 302. Forward bending motion of person 200 is another example of sagittal rotation motion 302 which may be a combination of movement from the person's hips and spine. Sagittal translation motion 304 corresponds to a translation motion orthogonal to sagittal plane 300. Sagittal translation motion 304 may also be referred to as moving right and/or left. Motions such as side bending or walking may have a component of sagittal translation motion 304. Frontal plane 310 divides person 200 into front and back portions, and when viewed orthogonally depicts a front or a back view of person 200 or exoskeleton 100. Frontal rotation motion 312 corresponds to a rotation along an axis orthogonal to frontal plane 310. Motions of person 200 such as side bending or walking may have a component of frontal rotation motion 312. Frontal translation motion 314 corresponds to a translation motion orthogonal to frontal plane 310. Frontal translation motion 314 may also be referred to as moving forward or backward. Flexion/extension of the person's hips, lower spine, and upper spine may have a component of frontal translation motion 314. Transverse plane 320 divides person 200 into upper and lower portions, and when viewed orthogonally depicts a top or bottom view of person 200 or exoskeleton 100. Transverse rotation motion 322 corresponds to a rotation along an axis orthogonal to transverse plane 320. Twisting and walking of person 200 may have a component of transverse rotation motion 322. Transverse translation motion 324 corresponds to a translation motion orthogonal to transverse plane 320. Transverse translation motion 324 may also be referred to as moving up or down. Walking may correspond to transverse translation motion 324. Misalignment between exoskeleton 100 and person 200 may correspond to any of the motions depicted in FIG. 3, particularly transverse translation motion 324, and sagittal rotation motion 302.

In some embodiments, tensile force direction 126 of flexible mechanism 130 most closely corresponds with frontal translation motion 314. For purposes of describing flexible mechanism 130, the planes and motions described in FIG. 3 are assumed to follow the upper trunk of person 200, such that when the person is bent forward in sagittal plane 300, frontal plane 310, and transverse plane 320 are also rotated to divide trunk 210 of person 200 and exoskeleton 100 similarly as is shown in FIG. 3. For example, frontal translation motion 314 is defined such that it remains orthogonal to person's trunk 210 or exoskeleton 100 in both the standing posture shown in FIG. 3 and FIG. 2, as well as the bent posture shown in FIG. 1. In some embodiments, the planes shown in FIG. 3 are rotated during bending motions of person 200 such that tensile force direction 126 remains substantially parallel with frontal plane translation motion 314. In some embodiments, where exoskeleton 100 supports a body part other than trunk 210 of person, frontal translation motion 314 is defined as the direction of tensile force direction 126. The definition of other rotation motions and translation motions can then be reoriented accordingly.

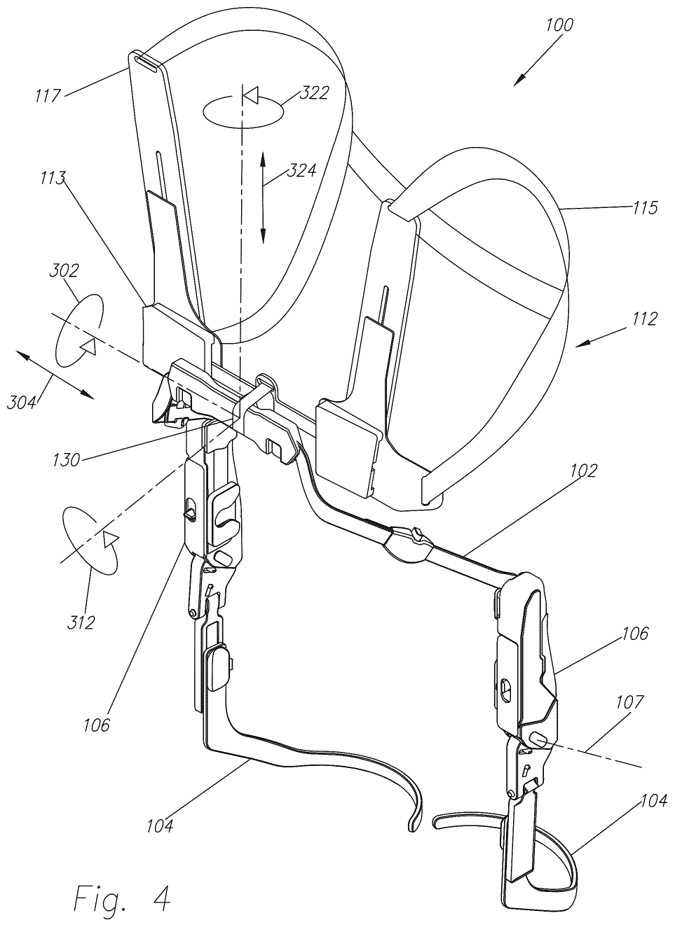

FIG. 4 shows a rear perspective view of exoskeleton 100 with flexible mechanism 130. Exoskeleton 100 is configured to generate supporting torque 120 between frame 102 and link 104 about joint 107, while flexible mechanism 130 is located approximately at a thoracic region of the person's spine. The planes and motions outlined in FIG. 3 are defined at the center of flexible mechanism 130 as shown in FIG. 4. Flexible mechanism 130 attaches human interface 112 to frame 102. Flexible mechanism 130 allows human interface 112 to move freely relative to frame 102 for sagittal rotation motion 302, sagittal translation motion 304, transverse rotation motion 322, transverse translation motion 324, and frontal rotation motion 312. In this embodiment, flexible mechanism 130 restricts frontal translation motion 314 between human interface 112 and frame 102.

Human interface 112 is configured to attach to trunk 210 of person 200 and transmit supportive torque 120 from exoskeleton 100 to person 200 as supportive force 122. Human interface 112 is configured to minimize compressive loads onto the spine as well as to minimize deflection in the plane of exoskeleton support. Human interface 112 is also configured to adjust to comfortably apply pressure to different-sized person 200. In some embodiments, human interface 112 encircles the chest and both shoulders of person 200. In the embodiment of FIG. 4, human interface 112 further comprises back plate 113 and shoulder straps 115. In some embodiments, back plate 113 is substantially rigid and is configured to receive the supporting forces and toques from frame 102 by means of the tensile forces in flexible mechanism 130. Back plate 113 comprises mounting points for a left and right shoulder strap 115 configured to at least partially encircle the shoulders or chest of person 200 and deliver supporting force 122 to trunk 210 of person 200. In this embodiment, supporting torque 120 from torque generator 106 is transferred from frame 102, through flexible mechanism 130 to back plate 113, and then distributed from back plate 113 to shoulder straps 115 which then apply supportive force 122 to trunk 210 of person 200.

In some embodiments, back plate 113 is arranged with sufficient rigidity between mounting points for shoulder straps 115 such that the mounting points are arranged with a defined width and height to prevent shoulder straps 115 from squeezing person 200 when forces are transmitted through them. In some embodiments, back plate 113 comprises at least four mounting points for shoulder straps 115, and shoulder straps 115 are configured to encircle both shoulders and chest of person 200. In other embodiments, back plate 113 comprises at least two mounting points for shoulder straps 115, and shoulder straps 115 are configured to encircle the chest of person 200 horizontally. In some embodiments, the defined width of back plate 113 between mounting points of shoulder straps 115 is adjustable to compensate for a varying with of person 200. In other embodiments, the defined height of back plate 113 between mounting points of shoulder straps 115 is adjustable to compensate for varying heights of person 200. The rigidity and geometry of back plate 113 allow tensile forces from flexible mechanism 130 to be distributed more evenly to person 200. Back plate 113 may be shaped to allow for exoskeleton 100 to be worn with PPE such as a safety harness. Back plate 113 may be shaped such that the area in the center of the back of person 200 is unobstructed to allow for the D-ring of a safety harness to be worn, as shown in FIG. 4.

In some embodiments, shoulder straps 115 comprise padding and inextensible webbing to most comfortably deliver the supporting force to trunk 210 of person 200. Shoulder straps 115 may adjust in length to best fit varying sizes of person 200. Shoulder straps 115 may comprise a number of secondary straps and or buckles to secure a right shoulder strap 115 and a left shoulder strap 115 across the chest of person 200. Shoulder straps 115 may comprise a section of elastic material to increase the comfort of person 200 during movement or breathing.

It is a goal of human interface 112 to minimize spinal compression forces on person 200. In some embodiments, shoulder plate 117 is arranged between back plate 113 and shoulder strap 115 of human interface 112. Shoulder plate 117 is configured to adjust the height of shoulder strap 115 mounting position relative to back plate 113. In use, shoulder plate 117 is adjusted such that the upper mounting position of shoulder strap 115 is located at the shoulder height of person 200. This allows forces transferred from shoulder plate 117 to shoulder strap 115 to be substantially in the horizontal direction relative to the chest of person 200. This minimizes downward forces on the person's trapezius muscles or spine that would occur if shoulder strap 115 mounting position was located substantially below the shoulder of person 200. In some embodiments, shoulder plate 117 is semi-rigid such that shoulder plate 117 can conform to the back of the person while transferring supportive forces. In some embodiments, shoulder plate 117 is resilient such that it returns to its original shape once bent, such as a leaf spring. Shoulder plate 117 may bend to accommodate for the bending of the person's spine above the level of the flexible mechanism 130. Shoulder plate 117 may be configured to provide support for bending motions of the person's spine above the level of the flexible mechanism 130, or to prevent a gap from occurring between human interface 112 and the person's back.

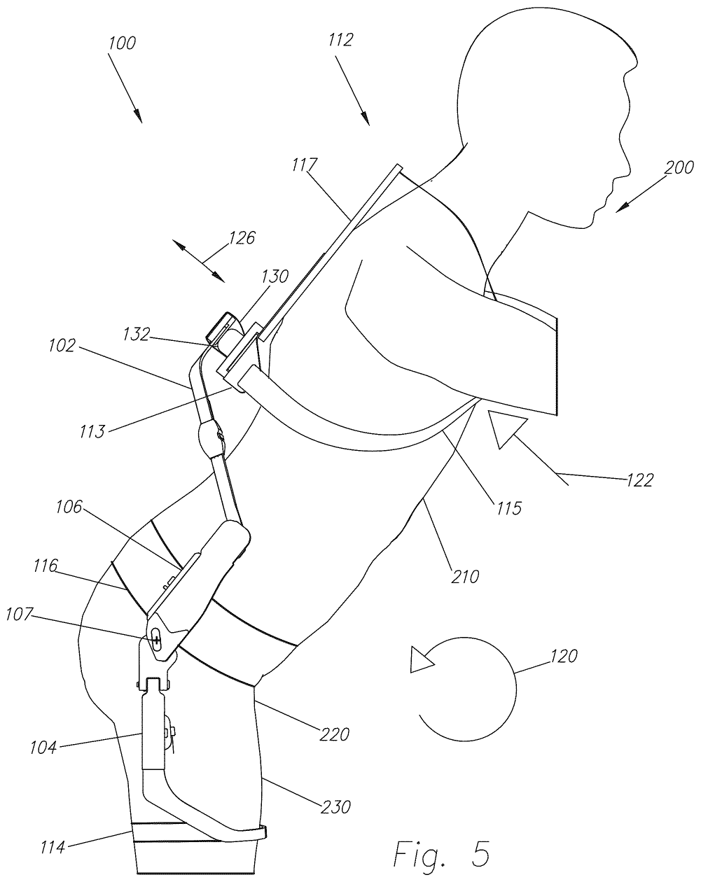

FIG. 5 shows one embodiment of exoskeleton 100 with flexible mechanism 130 on forward bending person 200. Torque generator 106 creates supporting torque 120 between frame 102 and link 104, which is applied to person's trunk 210 by human interface 112 and person's thigh 230 by link 104 or thigh strap 114. Flexible mechanism 130 transfers supporting torque 120 between frame 102 and human interface 112 through tensile forces along tensile force direction 126 such that supporting force 122 may be applied to person's trunk 210. In this view tensile force direction 126 corresponds to frontal translation motion 314 due to the fact that the center of motion of the forward bending of a person is coming from the person's hips or lower spine, located closer to joint 107 between frame 102 and link 104. In the embodiment of FIG. 5, the lower mounting point of shoulder strap 115 to back plate 113 is roughly in line with tensile force direction 126 of flexible mechanism 130. The upper mounting point of shoulder strap 115 to back plate 113 is located just above the person's shoulder to avoid compressive forces in the spine of person 200 as shoulder strap 115 tightens to deliver supporting force 122. In the embodiment of FIG. 5, flexible mechanism 130 is a length of webbing coupled to back plate 113 along two vertical locations, and is configured to at least partially encircle frame 102.

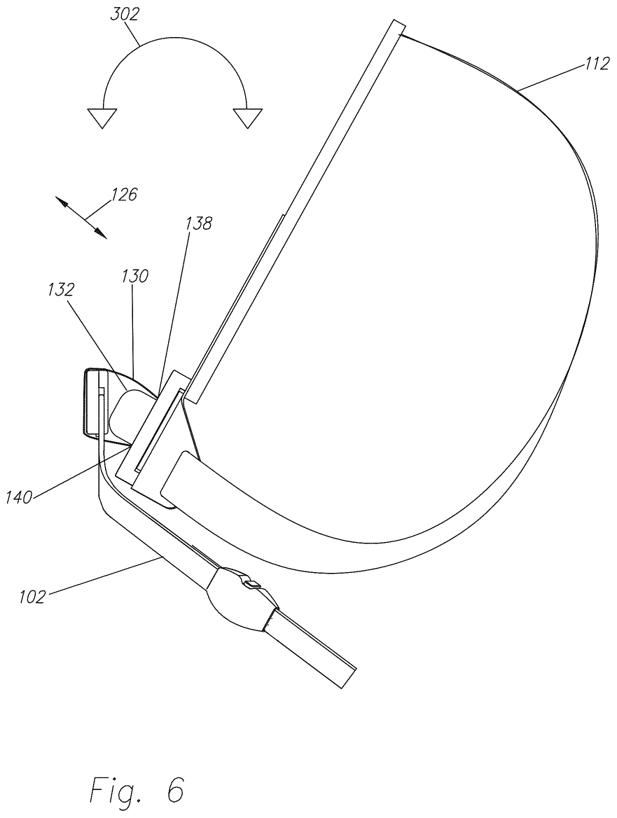

FIG. 6 depicts a side view of flexible mechanism 130 in sagittal rotation motion 302. In this view, it is visible that flexible mechanism 130 attaches to human interface 112 at first end 138 of flexible mechanism 130 and second end 140 of flexible mechanism 130 while partially encircling trunk frame 102. In this embodiment, the attachments to first end 138 of flexible mechanism 130 and to second end 140 of flexible mechanism 130 are arranged vertically on human interface 112. In this arrangement the tensile forces distributed by first end 138 of flexible mechanism 130 and second end 140 of the flexible mechanism 130 are equal. Flexible mechanism 130 allows human interface 112 to rotate relative to frame 102 along sagittal rotation motion 302 by sliding relative to frame 102. Sagittal rotation motion 302 occurs while flexible mechanism 130 continues to transfer tensile forces between human interface 112 and frame 102. During sagittal rotation motion 302 corresponding to forward flexion, the length of flexible mechanism 130 between first end 138 of flexible mechanism 130 and frame 102 will increase while the length of flexible mechanism 130 between second end 140 of flexible mechanism 130 and frame 102 will decrease. During this process, tensile forces are distributed between frame 102 and human interface 112 by both first end 138 and second end 140 of flexible mechanism 130. Tensile force direction 126 between first end 138 and frame 102 may be slightly different than tensile force direction 126 between second end 140 and frame 102, but when added together remain roughly aligned with frontal translation motion 314. In some embodiments, the materials of flexible mechanism 130 and trunk frame 102 are chosen to minimize friction to smooth the motion and increase the life of exoskeleton 100.

In some embodiments, human interface 112 further comprises cam element 132. Cam element 132 may be configured to provide a rolling or sliding surface between frame 102 and human interface 112. Cam element 132 may be configured to adjust the center of rotation between frame 102 and human interface 112 for one or multiple rotation directions. In the embodiment of FIG. 6, cam element is configured to provide a rolling contact surface between frame 102 and human interface 112 to facilitate sagittal rotation motion 302. In this embodiment, cam element creates a center of rotation for sagittal rotation motion 302 approximately aligned with the spine of person 200. In some embodiments, cam element 132 creates a center of rotation that reduces the amount of slack needed in flexible mechanism 130 to allow for smooth sagittal rotation motion 302.

In some embodiments, sagittal rotation motion 302 between human interface 112 and frame 102 helps to accommodate for flexion of the spine of person 200 above support joint 107 such that human interface 112 remains static with respect to person's trunk 210. Without sagittal rotation motion 302, flexion of the spine of person 200 above the support rotational axis could cause a gap between the upper portion of human interface 112 and person 200, the lower portion of human interface 112 to dig into a person, slack in the lower portion of shoulder straps 115 or excess tension in the upper portion of shoulder straps 115, all of which create discomfort for person 200. Flexible mechanism 130 allows flexion of the spine of person 200 above support joint 107 without substantial change in the pressure distribution of human interface 112 on person's trunk 210.

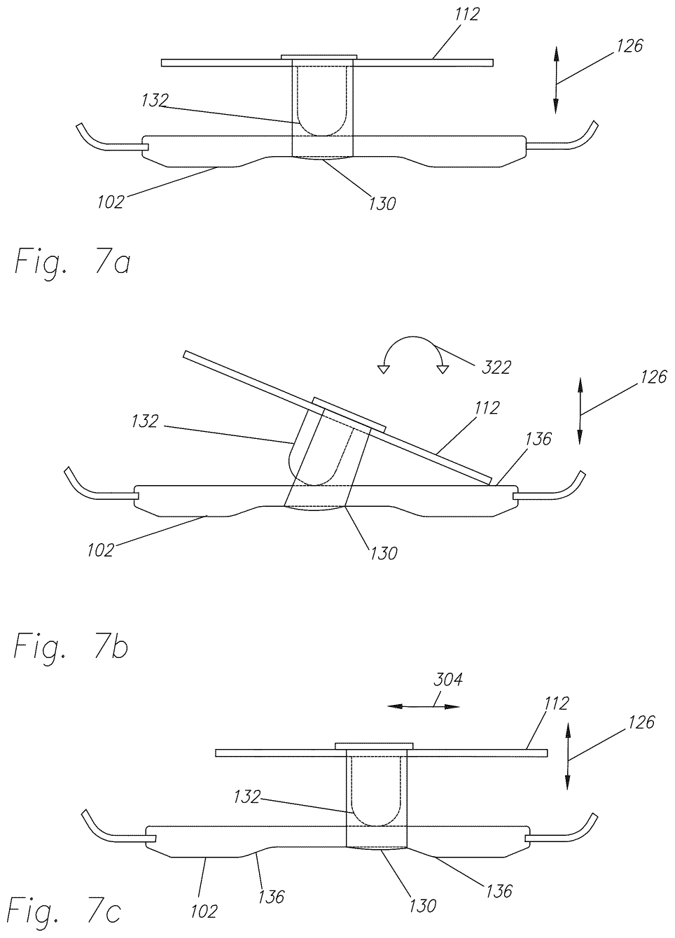

FIGS. 7a-7c depict a top view of flexible mechanism 130 in a variety of positions. FIG. 7a shows flexible mechanism 130 in a neutral position as it would be if person 200 was standing as in FIG. 2. FIG. 7b shows flexible mechanism 130 providing a transverse rotation motion 322 between frame 102 and human interface 112. To provide transverse rotation motion 322, flexible mechanism 130 may twist. Human interface 112 may further comprise cam element 132 configured to control transverse rotation motion 322 by providing a rolling contact surface between human interface 112 and frame 102. Cam element 132 may further serve to create a space between human interface 112 and frame 102 to allow for the needed rotational motion before a hard stop occurs. Frame 102 may further comprise a hard stop 136 configured to limit the range of motion of human interface 112. In the configuration of FIG. 7b, hard stop 136 is created by the contact point between human interface 112 and frame 102 along transverse rotation motion 322. FIG. 7c shows flexible mechanism 130 providing sagittal translation motion 304. In some embodiments, flexible mechanism 130 is configured to slide along frame 102 to provide sagittal translation motion 304. In some embodiments, frame 102 comprises hard stop 136 in both directions of sagittal translation motion 304 to limit the motion of human interface 112 relative to frame 102. Hard stop 136 may be formed by a bulge in frame 102 that flexible mechanism 130 is not able to pass over.

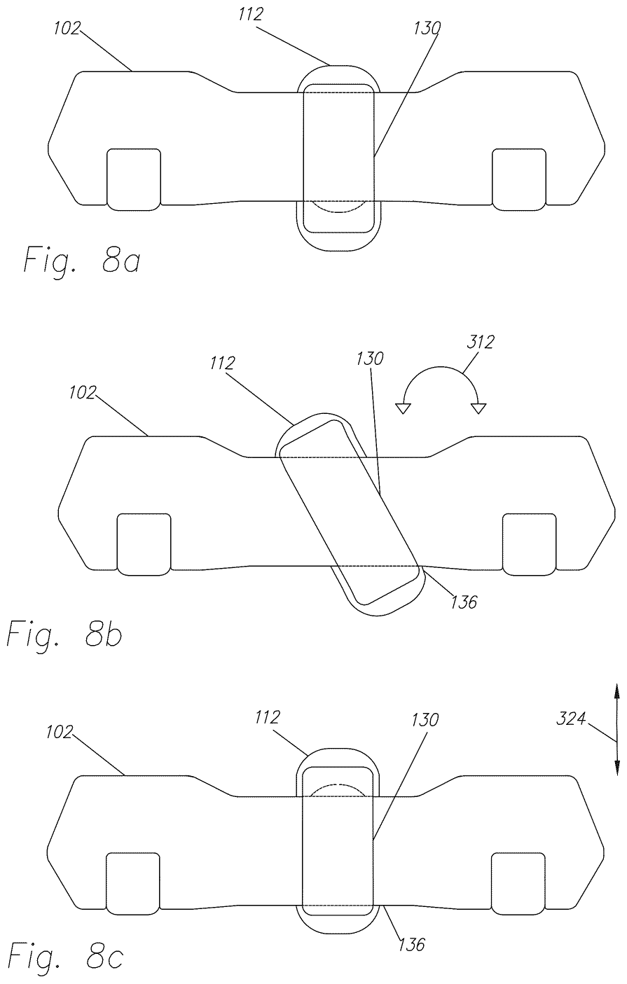

FIGS. 8a-8c depicts a back view of flexible mechanism 130 in a variety of positions. For simplicity, only a small portion of human interface 112 is shown. FIG. 8a shows flexible mechanism 130 in a neutral position as in FIG. 7a and FIG. 2. FIG. 8b shows flexible mechanism 130 providing a frontal rotation motion between frame 102 and human interface 112. In some embodiments, flexible mechanism 130 twists or deforms to allow frontal rotation motion 312. Hard stop 136 may be formed by the dimensions of frame 102 compared to the vertical distance between the mounting points of flexible mechanism 130 on human interface 112. FIG. 8c shows flexible mechanism 130 providing for transverse translation motion 324 between human interface 112 and frame 102. Hard stop 136 may similarly be formed by the dimensions of frame 102 compared to the vertical distance between the mounting points of flexible mechanism 130 on human interface 112.

In the embodiments of FIG. 4 through FIGS. 8a-8c flexible mechanism 130 transfers forces in frontal translation motion 314 while allowing free motion in all other directions. Cam element 132 may aid in the motion of one or all of the motions including sagittal rotation motion 302, sagittal translation motion 304, frontal rotation motion 312, transverse rotation motion 322, or transverse translation motion 324. Similarly, the geometry of human interface 112 and frame 102, as well as the mounting locations of flexible mechanism 130 may all interact to form hard stop 136 to limit one or all of the motions including sagittal rotation motion 302, sagittal translation motion 304, frontal rotation motion 312, transverse rotation motion 322, or transverse translation motion 324.

One skilled in the art may appreciate that the combination of motions including sagittal rotation motion 302, sagittal translation motion 304, frontal rotation motion 312, transverse rotation motion 322, or transverse translation motion 324 may result in a variety of complex motions. For example, the combination of sagittal translation motion 304 and frontal rotation motion 312 may result in allowing, human interface 112 to rotate relative to frame 102 in frontal plane 310 about a joint center near the person's lower back, far outside of the physical location of flexible mechanism 130. This motion is of particular importance during walking, when person's trunk 210 rhythmically moves relative to person's hips 220 with a complex rotation about multiple joint centers corresponding to spinal vertebrae. Similarly, the combination of sagittal rotation motion 302 and transverse translation motion 324 may allow human interface 112 to rotate relative to frame 102 in sagittal plane 300 about a joint center near the person's lower back, far outside of the physical location of flexible mechanism 130. This motion is of particular importance during forward bending, as person's trunk 210 moves relative to person's hips 220 with a complex rotation about multiple joint centers corresponding to spinal vertebrae. The kinematic redundancy of flexible mechanism 130 allows exoskeleton 100 to comfortably follow the complex motions of person 200, as well as allow for unique differences in motion between various users without compromising the delivery of the supportive torques and forces.

One skilled in the art may also appreciate that a lack of tensile forces in flexible mechanism 130, corresponding to a state of exoskeleton 100 that is not generating a supportive torque 120, allows for increased motion between human interface 112 and frame 102 due to slack that may be present in flexible mechanism 130. This allows person 200 greater freedom during non-bending postures. In some embodiments, the length of flexible mechanism 130 may be adjustable to change the range of motion of human interface 112 relative to frame 102. In other embodiments, the mounting locations of flexible mechanism 130 may be adjustable to change the range of motion of human interface 112 relative to frame 102.

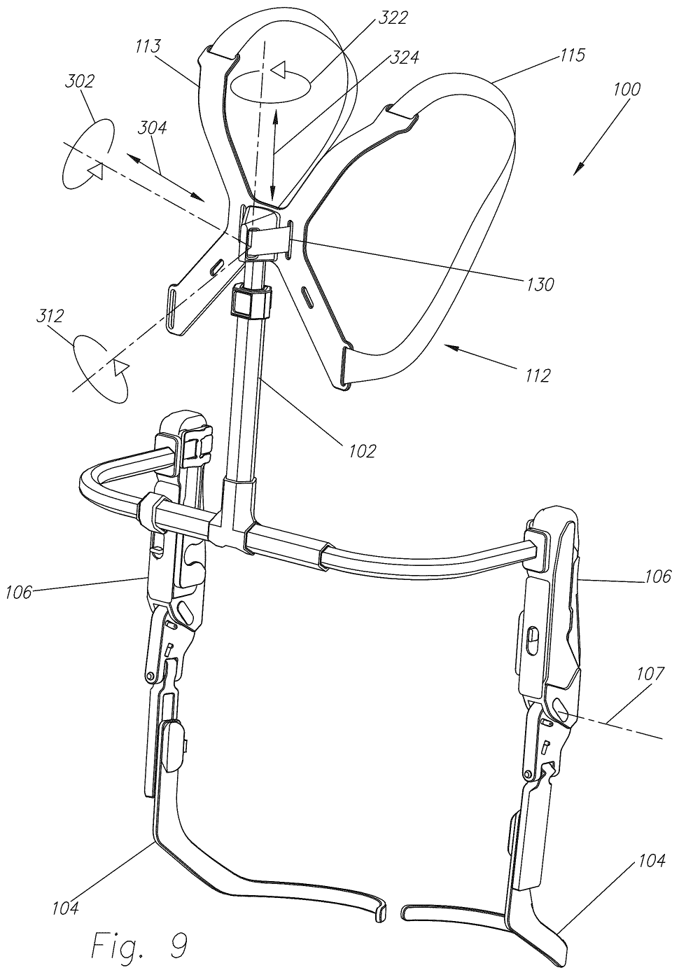

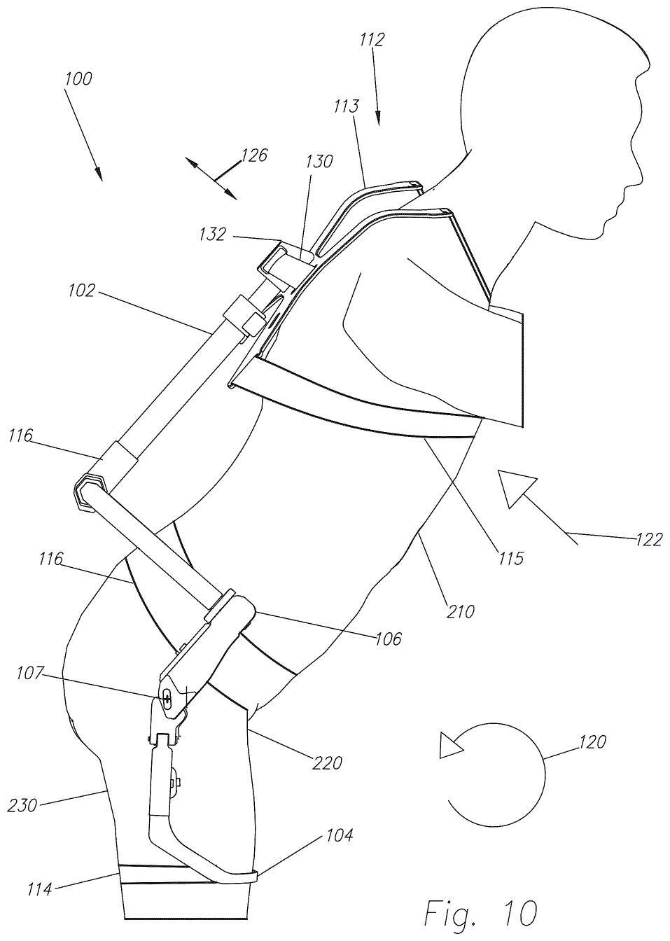

FIG. 9 through FIG. 13 show an alternate embodiment of exoskeleton 100 with flexible mechanism 130. The primary difference in the alternate embodiment is the shape of frame 102, human interface 112, and cam element 132 and how they interact to allow the degrees of freedom provided by flexible mechanism 130. As shown in FIG. 9, frame 102 comprises a single vertical spine portion behind person 200 and a horizontal hip portion to connect to torque generator 106. as opposed to frame 102 in FIG. 4 that comprises a horizontal spine portion behind person 200 that connects to torque generator 106 through a diagonal side portion. Flexible mechanism 130 of FIG. 9 is connected to human interface 112 at two horizontally opposed positions and is looped around frame 102. This creates some differences is movement as will be explained below. FIG. 10 shows a side view of the alternate embodiment of exoskeleton 100 with flexible mechanism 130 on forward bending person 200. Flexible mechanism 130 may be approximately centered between the upper and lower mounting location of shoulder straps 115 along back plate 113. A centered location of flexible mechanism 130 along human interface 112 may serve to balance the forces in human interface 112 to aid in the comfort of the person and reduce unwanted rotations of flexible mechanism 130. A centered location of flexible mechanism 130 along human interface 112 may further serve to optimize tensile force direction 126 to remain substantially orthogonal to frame 102 about joint 107 throughout the range of motion of human interface 112 relative to frame 102. In the embodiment of FIG. 9 frame 102 further comprises cam element 132. Cam element 132 comprises a slot through which flexible mechanism 130 passes such that flexible mechanism 130 at least partially encircles cam element 132. Cam element 132 serves to constrain flexible mechanism 130, create a rolling or sliding contact surface between frame 102 and human interface 112, and create sufficient space between frame 102 and human interface 112 to facilitate the needed range of motion.

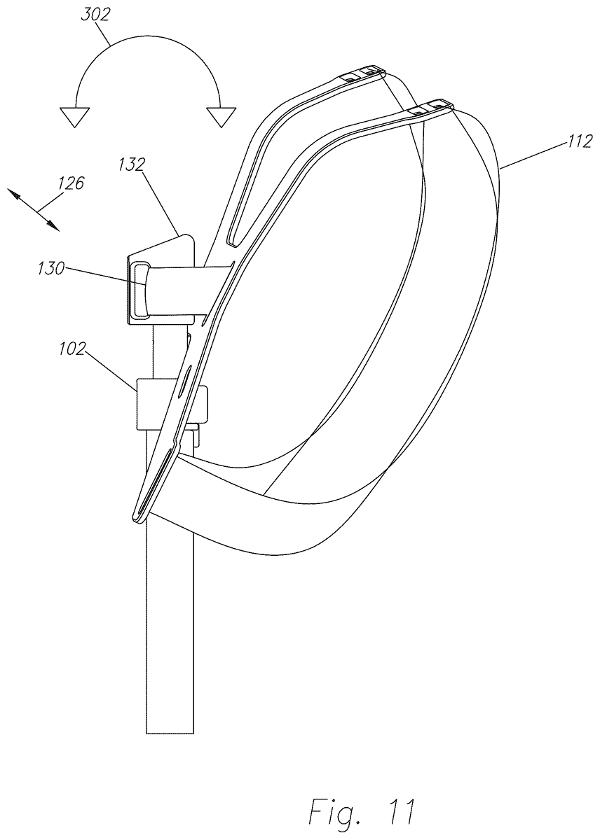

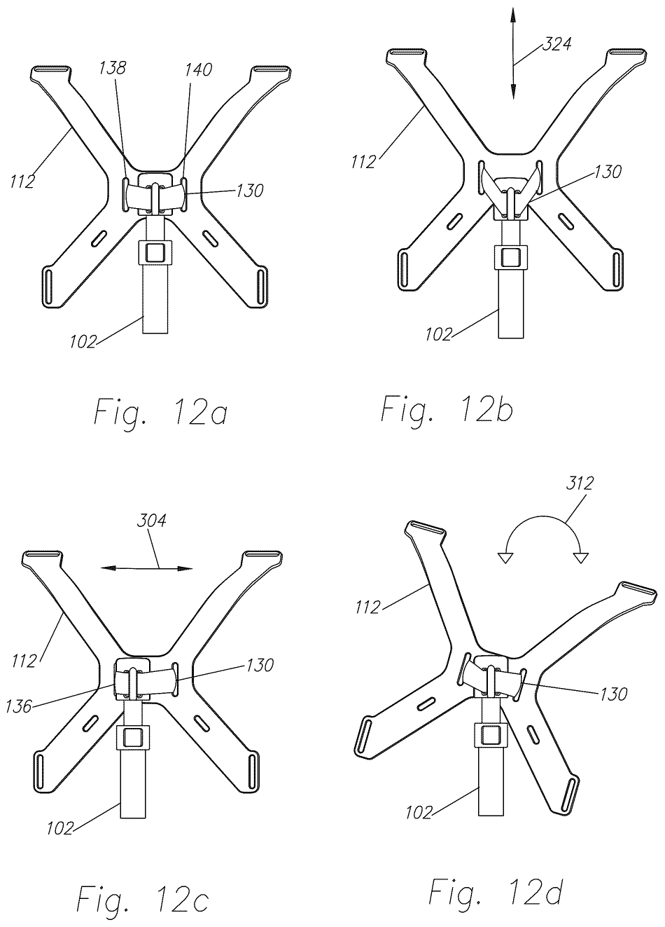

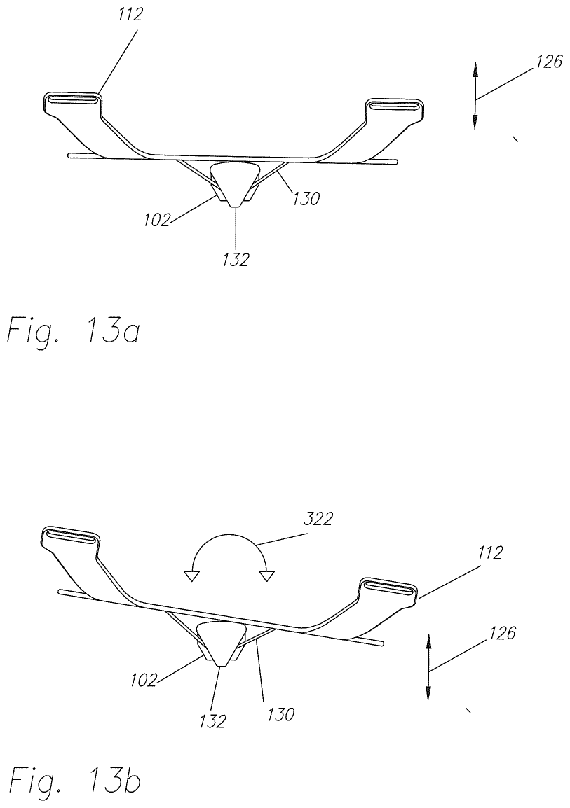

FIG. 11 shows a side view of flexible mechanism 130 providing sagittal rotation motion 302 between human interface 112 and frame 102. Flexible mechanism 130 twists while cam element 132 provides a rolling contact between frame 102 and human interface 112. FIG. 12 depicts a top view of flexible mechanism 130 in (a) a neutral position (b) transverse translation motion 342 (c) sagittal translation motion 304 and (d) frontal rotation motion 312. FIG. 13 depicts a back view of flexible mechanism 130 in (a) a neutral position and (b) transverse rotation motion 322. During transverse translation motion 324 and frontal rotation motion 312, flexible mechanism 130 twists while human interface 112 slides relative to the frame. During sagittal translation motion 304 and transverse rotation motion 322 flexible mechanism 130 slides within the slot for cam element 132. Cam element 132 comprises curved surfaces to help define transverse rotation motion 322 and sagittal rotation motion 302. Similar to prior embodiments, the range of motion provided by flexible mechanism 130 may be defined by cam element 132, the length of flexible mechanism 130, and the geometry of frame 102 and human interface 112. It can be seen by one skilled in the art that flexible mechanism 130 can provide five degrees of freedom while transferring tensile forces for frontal translation motion 314 corresponding to the forward bending of person 200.

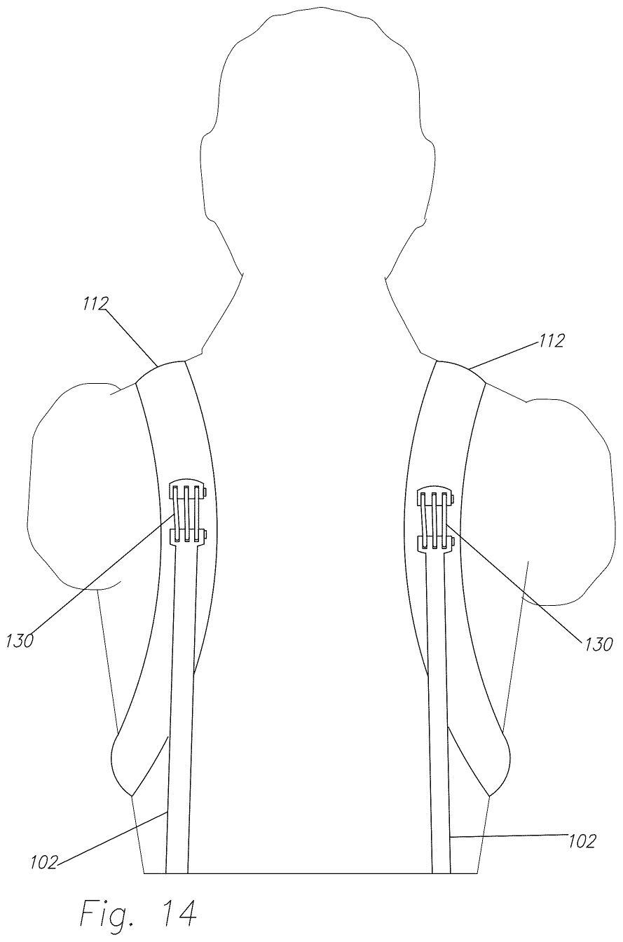

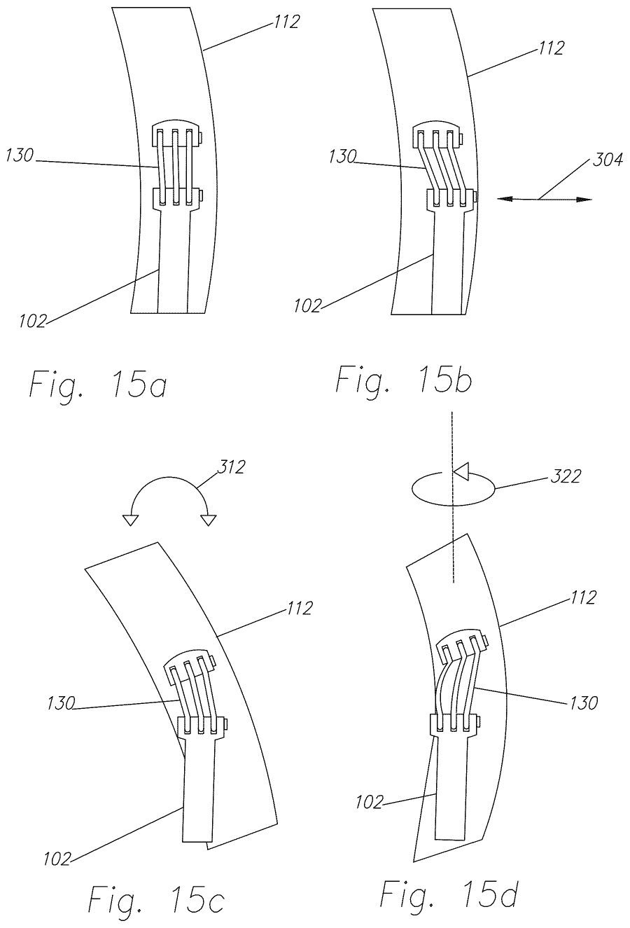

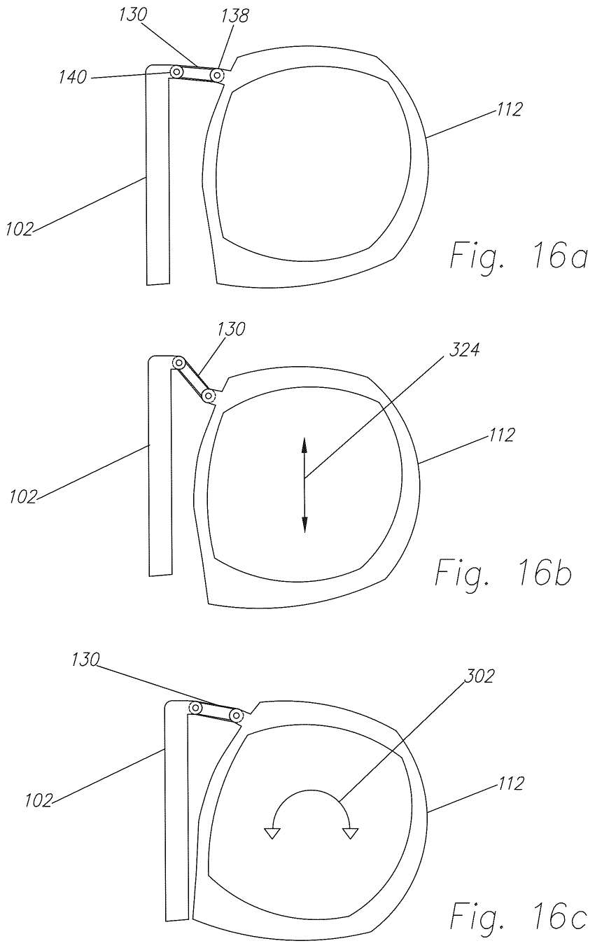

FIG. 14 through FIG. 16 show an alternate embodiment of exoskeleton 100 with flexible mechanism 130. FIG. 14 shows a rear view of exoskeleton 100 with flexible mechanism 130. In this embodiment frame 102 comprises two separate vertical spine sections behind person 200. For each spine section of frame 102, flexible mechanism 130 connects to human interface 112. In this embodiment, flexible mechanism 130 is coupled to human interface 112 from first end 138 and to frame 102 from second end 140. In some embodiments, flexible mechanism 130 is a loop that at least partially encircles a portion of frame 102 and human interface 112. In other embodiments, flexible mechanism 130 may be coupled to a pivot point on frame 102 or human interface 112. In some embodiments, flexible mechanism 130 comprises multiple loops or lines between frame 102 and human interface 112. FIG. 15 depicts a rear view of flexible mechanism 130 in (a) a neutral position (b) sagittal translation motion 304 (c) frontal rotation motion 312 and (d) transverse rotation motion 322. FIG. 16 depicts a side view of flexible mechanism 130 in (a) a neutral position (b) transverse translation motion 324 and (c) sagittal rotation motion 302. For each motion of human interface 112 relative to frame 102, flexible mechanism 130 twists, rotates, or slides relative to frame 102 or human interface 112. For frontal rotation motion 312 or transverse rotation motion 322 a portion of flexible mechanism 130 may go slack while a remaining portion of flexible mechanism 130 remains taught to transfer tensile forces.

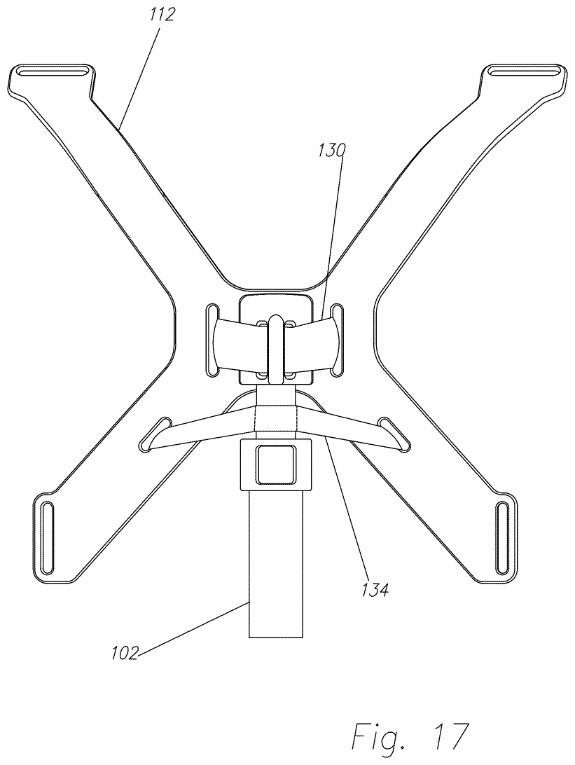

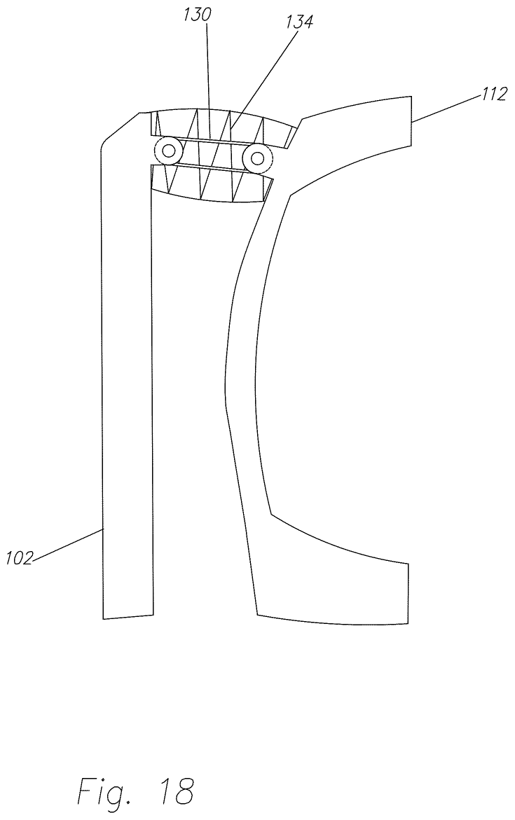

In some embodiments shown in FIG. 17 and FIG. 18, exoskeleton 100 further comprises centering element 134 that is configured to reduce excess motion between human interface 112 and frame 102 when flexible mechanism 130 is not transferring tensile loads. In other embodiments, centering element 134 biases human interface 112 into a neutral, or centered, position relative to frame 102. When human interface 112 twists, side bends, or moves off-center of frame 102, centering element 134 applies a force to re-center human interface 112. In some embodiments, centering element 134 allows exoskeleton 100 to be more easily put on and taken off by reducing the excess motion of human interface 112. In other embodiments, centering element 134 biases a person 200 posture into a neutral position when wearing the exoskeleton. In some embodiments, centering element 134 should allow the full motion of human interface 112 relative to frame 102. In other embodiments, centering element 134 acts to hard stop the motion of human interface 112 relative to frame 102.

FIG. 17 shows one embodiment of exoskeleton 100 comprising centering element 134 in a neutral position. Centering element 134 is attached to frame 102 at a middle portion and to human interface 112 along each of two equally long side portions. For any motion of human interface 112 relative to frame 102, centering element 134 will stretch, either symmetrically or asymmetrically, to return human interface 112 to a neutral position. For example, during transverse rotation motion 322 one end of centering element 134 will stretch while the other end of centering element 134 will go slack. The stretch end of centering element 134 will then apply a force to human interface 112 in the opposite direction of transverse rotation motion 322. In another example, for a transverse translation motion 324, both ends of centering element 134 will stretch and create a force to move human interface 112 in the opposite direction of transverse translation motion 324. Centering element 134 may be comprised of coil springs, elastic chord, elastic webbing, or any other resilient material. In other embodiments not shown flexible mechanism 130 may be partially made of elastic material and act as a centering element.

FIG. 18 shows a second embodiment of exoskeleton 100 comprising centering element 134 in a neutral position. Centering element 134 comprises a spring situated between frame 102 and human interface 112. Centering element 134 spring may surround flexible mechanism 130 and be mounted such that centering element 134 can apply a force between human interface 112 and frame 102 for any rotational or translational motion of human interface 112. Centering element 134 may bias human interface 112 away from frame 102 until a tensile force is created in flexible mechanism 130. In other embodiments, centering element 134 is a resilient block positioned between human interface 112 and frame 102. The resilient block may be made of foam or similar material with spring or damping properties. Centering element 134 may be a coil spring, a flexible rod made with materials such as PEEK, and carbon fiber. In some embodiments, flexible mechanism 130 is configured to sit inside of centering element 134.

The freedom of motion between human interface 112 and frame 102 by flexible mechanism 130 likewise contributes to increased comfort for person 200 during walking and other non-bending tasks. Flexible mechanism 130 allows human interface 112 to translate and rotate relative to frame 102 such that the trunk of person 200 moves naturally during gait as it is not impacted by the rigidity of the exoskeleton spine. Flexible mechanism 130 also reduces vibration or impulses from the legs of person 200 during walking or other motions from traveling along frame 102 to person's trunk 210. Flexible mechanism 130 also allows for improved comfort during awkward postures as it affords a greater range of motion.

It is important that flexible mechanism 130 optimizes force transmission to the human body along tensile force direction 126. This ensures that when the exoskeleton is active, only support force 122 is orthogonal to the spine of person 200 is transmitted, and not forces parallel to the spine which may contribute to spinal compression. Similarly, flexible mechanism 130 optimizes the general distribution of the exoskeleton's weight on the human body. As it allows translation between human interface 112 and frame 102, it ensures that the weight of the exoskeleton is completely carried on the person person's hips 220. This minimization of weight applied to the shoulders gives the subjective feeling that the system is lighter—as the human hips are more capable of supporting vertical loads than the human shoulder girdle.

Flexible mechanism 130 between frame 102 and human interface 112 is able to sufficiently couple the human interface 112 to frame 102 to provide back support, yet allow a substantial range of motion for the user in twisting, side-bending, and allows human interface 112 to tilt along with the user's upper back when bending. It is a simple and cost-effective coupling between human interface 112 and frame 102, which. In some embodiments, flexible mechanism 130 may utilize a triglide (3 bar metal slider) and/or hook and loop fasteners to secure human interface 112 to frame 102. Flexible mechanism 130 secures to the two slots on back plate 113, and loops through frame 102. For the best overall fit, it is critical that flexible mechanism 130 between frame 102 and human interface 112 is located near the thoracic region of its user. This central location on the spine provides the best alignment between the human spinal range of motion and that of flexible mechanism 130 between human interface 112 and frame 102.

It can be understood by one skilled in the art that flexible mechanism 130 may be used in other locations on the human body in conjunction with exoskeletons that provide varying types of supportive forces. For instance, on exoskeleton 100 flexible mechanism 130 may also be utilized to connect link 104 to a textile thigh cuff to optimize forces applied to thigh 230 of person 200. In this embodiment, tensile force direction 126 is approximately orthogonal to the thigh of person 200 and frontal translation motion 314 is defined as parallel to tensile force direction 126. On a shoulder-supporting exoskeleton, flexible mechanism 130 may similarly be used to attach a rigid arm link to a textile arm cuff. In this embodiment, tensile force direction 126 is approximately orthogonal to the arm of person 200 and frontal translation motion 314 is defined as parallel to tensile force direction 126. The key element is that the flexible mechanism 130 transmits tensile forces between a frame and an interface on the body of person 200 while allowing for free motion in all other directions. In some embodiments, human interface 112 may be referred to as interface 112 and is configured to couple to the body of person 200, such as an arm, leg, neck, head, upper arm, forearm, foot, hand, thigh, or shank. In some embodiments, frame 102 may be configured to move approximately in unison with person's foot, shank, thigh, hips, trunk, upper arm, forearm, hand, neck, head, arm, or leg. In some embodiments, link 104 may be configured to move approximately in unison with person's foot, shank, thigh, hips, trunk, upper arm, forearm, hand, neck, head, arm, or leg. Similarly some embodiments, torque generator 106 may be configured to generate a supportive torque 120 between link 104 and frame 102 to support a person's foot, shank, thigh, hips, trunk, upper arm, forearm, hand, neck, head, arm, leg, or other body part or joint. For other locations of support, frontal plane 310 may be referred to as support plane 310 with support translation motion 314 and support rotation motion 312. Support plane 310 is oriented such that it is orthogonal to tensile force direction 126. Sagittal plane 300 may be referred to as a first orthogonal plane, with a first translation motion and a first rotation motion. Transverse plane 320 may be referred to as second orthogonal plane 320 with a second rotation motion and a second translation motion.

In some embodiments, not shown, the textile interface may further facilitate a rapid attachment or detachment of frame 102 relative to the human interface 112. This feature may be used to aid in donning the exoskeleton, or to allow for multiple users to have their own human interface 112 and share a single frame 102, the key component that generates support force 122. The rapid attachment of frame 102 relative to human interface 112 may utilize configurations with velcro, magnets, a mechanical locking mechanism, slots for threading the textile interface through, or any combination.

1. An exoskeleton configured to be worn by a person to reduce muscle forces in a back of the person during forward lumbar flexion, the exoskeleton comprising:
a frame configured to transfer a supporting torque;
a human interface configured to couple to a trunk of the person; and
a flexible mechanism configured to transfer tensile forces between the frame and the human interface, wherein when the frame transfers the supporting torque, the flexible mechanism transfers the supporting torque to the human interface by means of the tensile forces.

2. The exoskeleton of claim 1, wherein the flexible mechanism restricts a frontal translation motion between the human interface and the frame.

3. The exoskeleton of claim 1, wherein the flexible mechanism allows for a sagittal translation motion between the human interface and the frame.

4. The exoskeleton of claim 1, wherein the flexible mechanism allows for a transverse translation motion between the human interface and the frame.

5. The exoskeleton of claim 1, wherein the flexible mechanism allows for one or more of:
a sagittal translation motion between the human interface and the frame,
for a transverse translation motion between the human interface and the frame,
a sagittal rotation motion between the human interface and the frame,
a frontal rotation motion between the human interface and the frame, and
a transverse rotation motion between the human interface and the frame.

6. The exoskeleton of claim 1, wherein, when the person bends forward and the frame transfers the supporting torque, the flexible mechanism restricts a frontal translation motion to transfer the supporting torque from the frame to the human interface, and the flexible mechanism allows for a sagittal rotation motion and a transverse translation motion between the frame and the human interface reduce relative motion between the human interface and the trunk of the person.

7. The exoskeleton of claim 1, wherein, when the person bends forward and the frame transfers the supporting torque, the flexible mechanism restricts a frontal translation motion to transfer the supporting torque from the frame to the human interface, and the flexible mechanism provides a frontal rotation motion and a sagittal translation motion to allow the person to side bend.

8. The exoskeleton of claim 1, wherein, when the person bends forward and the frame transfers the supporting torque, the flexible mechanism restricts a frontal translation motion to transfer the supporting torque from the frame to the human interface, and the flexible mechanism provides a transverse rotation motion to allow the person to twist.

9. The exoskeleton of claim 1, wherein the flexible mechanism provides for a range of motion selected from the group consisting of sagittal translation, transverse translation, frontal rotation, and transverse rotation to allow for hips of the person to move relative to the trunk of the person during walking.

10. The exoskeleton of claim 1, wherein, when the person performs a walking motion, the frame follows the motion of hips of the person, and the human interface moves in a sagittal translation motion to follow the motion of the trunk of the person.

11. The exoskeleton of claim 1, wherein when the person performs a side bending motion, the frame follows the motion of hips of the person, and the human interface moves in a frontal rotation motion and a sagittal translation motion to follow the motion of the trunk of the person.

12. The exoskeleton of claim 1, wherein, when the person performs a twisting motion, the frame follows the motion of hips of the person, and the human interface moves in a transverse rotation motion to follow the motion of the trunk of the person.

13. The exoskeleton of claim 1, wherein the flexible mechanism comprises a length of inextensible webbing, rope, or cable.

14. The exoskeleton of claim 1, wherein the flexible mechanism is coupled to the frame from its first end and is coupled to the human interface from its second end.

15. The exoskeleton of claim 1, wherein the flexible mechanism is coupled to the human interface at both ends and is configured to at least partially encircle the frame, and wherein flexible mechanism is configured to slide relative to the frame.

16. The exoskeleton of claim 1, wherein the human interface comprises a rigid back plate configured to be coupled to the flexible mechanism, and a shoulder strap coupled to the rigid back plate configured to at least partially encircle the shoulders or chest of the person.

17. The exoskeleton of claim 1, wherein the human interface comprises a rigid back plate configured to be coupled to the flexible mechanism, and a shoulder strap coupled to the rigid back plate at an upper location and a lower location configured to at least partially encircle the shoulders or chest of the person, wherein the flexible mechanism is coupled to the rigid back plate approximately halfway between the upper location and the lower location.

18. The exoskeleton of claim 1, wherein the human interface comprises a rigid back plate configured to be coupled to the flexible mechanism, and a shoulder strap coupled to the rigid back plate at an upper location and a lower location configured to at least partially encircle the shoulders or chest of the person, wherein the flexible mechanism is coupled to the rigid back plate approximately inline with the lower location.

19. The exoskeleton of claim 1 further comprising a cam element coupled to either the human interface or the frame, wherein the cam element is configured to create a space and provide a rolling contact surface between the human interface and the frame to allow for rotation motions.

20. The exoskeleton of claim 1 further comprising a centering element coupled between the frame and the human interface, wherein the centering element is configured to apply a force to bring the human interface to a neutral position relative to the frame.
